(19) United States
(12) Patent Application Publication
(10) Pub. No.: US 2003/0013083 A1
(43) Pub. Date: Jan. 16, 2003

(54) Particle analysis as a detection system for particle-enhanced assays

(76) Inventors: Tenlin S. Tsai, Mountain View, CA (US); Renliang Xu, Pembroke Pines, FL (US)

Correspondence Address: BECKMAN COULTER INC, 4300 NORTH HARBOR BOULEVARD, P O BOX 3100, FULLERTON, CA, 928343100

(21) Appl. No.: 09/906,511

(22) Filed: Jul. 16, 2001

(51) Int. Cl. C12Q 1/70; C12Q 1/68; G01N 33/543

(52) U.S. Cl. 435/5; 435/6; 436/518

(57) ABSTRACT

This invention provides an improved particle-enhanced assay for determining the concentration of any of a wide range of analytes with a high degree of specificity, accuracy, and sensitivity, primarily by improving the measurement of particle aggregates. Analyte concentration is determined by effecting a particle-enhanced reaction in an assay medium and measuring the distribution of different sized aggregated particles in the reaction mixture by polarization intensity differential scattering. The particle size distribution is then compared with a standard curve to determine the concentration of the analyte in the sample.

### FIELD OF THE INVENTION

This invention relates to the field of diagnostics, and more particularly to a sensitive particle-enhanced assay for measuring the concentration of an analyte of interest in a sample. The particle analysis of the reaction mixture is determined using the Polarization Intensity Differential Scattering (PIDS) technique. The particle size distribution is then correlated with the concentration of analyte in the sample.

### BACKGROUND OF THE INVENTION

A number of commonly used clinical chemistry assays detect and quantify various substances present in test samples, such as physiological fluids through immunological reactions, resulting in the formation of immune complexes. An immune complex forms when two reaction partners, each with a specific binding affinity for the other, are combined in a suitable assay medium. The reaction partners can be an antigen and a specific binding partner for the antigen, such as an antibody. Generally, one of the reaction partners, known as the analyte, is present in an unknown amount in the test sample. In particle-enhanced immunoassays, one of the reaction partners is immobilized on insoluble particles, such as latex particles. Once initiated, the particle-enhanced immunological reaction results in the formation of particle aggregates, e.g., antibody-antigen complexes that are insoluble in the assay medium. In a direct particle-enhanced assay, the extent of particle aggregation is directly proportional to the concentration of the analyte in the sample.

Particle aggregates can be detected in any of a variety of ways. For example, if the particle aggregates are large enough, they will become capable of scattering light, which can be detected by photometric immunoassay techniques. The presence of particle aggregates in the assay medium can change optical properties, such as light-scattering and light absorption properties of the assay medium, by attenuation of incident light energy. These changes can be detected by various light-scattering methods. Static or “classical” light-scattering methods are based upon illumination of a sample containing the particles, followed by the measurement of the intensity of scattered light at several predetermined angles. The intensity of light scattered from a particle is a function of the size of the particle, the wavelength of incident light, and the angle at which the scattered light is collected relative to the incident light. This method of particle sizing, based upon the angular dependence of the scattered light intensity, can be employed to determine the size distribution of a group of particles. These changes can be detected by an appropriate photometer. The photometer can be calibrated to permit detection and quantification of the analyte. Calibration is typically carried out by conducting the particle-enhanced reaction with known amounts of an analyte of interest in an assay medium to derive a standard or calibration curve. The calibration curve can show a level of light attenuation, for example, light absorption, by the assay medium versus the amount of analyte present per unit volume of medium. (See, for example, U.S. Pat. Nos. 4,080,264 and 4,851,329 to Cohen et al.; P. L. Masson, et al, “Particle Counting Immunoassay (PACIA), Methods in Enzymology,” Vol. 74, pp. 106-139 (1981) Academic Press, S. P. Colowick and N. O. Kaplan, eds.; T. A. Wilkins, et al., “Immunoassay by Particle Counting, Complementary Immunoassays,” pp. 227-240 (1988) W. P., ed.).

Examples of photometric techniques for measuring light-scattering by particle aggregates include nephelometry and turbidimetry. Nephelometric methods measure the light scattered by a suspension of particles or reflected towards a detector that is not in the direct path of light. In contrast, turbidimetric methods measure the reduction of light transmitted through the suspension of particles, where a light detector is placed in the light path. The reduction is caused by reflection, scatter, and absorption of the light by the particles. However, the requirement of producing large immune complexes has limited the applicability of nephelometric and turbidometric immunoassays to high molecular weight molecules, such as proteins, that possess several epitopes (i.e., antibody-binding sites). In particular, many haptens, such as therapeutic agents, have only a single epitope and, as such, are incapable of forming the large immune complexes needed for such immunoassays.

U.S. Pat. No. 4,080,264, issued to Cohen et al,. discloses a particle-based immunoassay utilizing photon correlation spectroscopy to determine the concentration of an antibody or antigen of interest in a test sample. The presence of a target analyte in the medium leads to aggregation and an increase in particle size, which is related to analyte concentration. Direct measurement of the mean diffusion coefficient of the particles can be related to particle size through the Einstein and Stokes equations. However, photon correlation spectroscopy is known to have low resolution and is unable to accurately detect small amounts of particle aggregates. Thus, this method has a substantial lack of sensitivity.

U.S. Pat. No. 4,174,952, issued to Cannell et al,. discloses a particle-based immunoassay in which the ratio of scattered light intensity at two different angles, the so-called anisotropy ratio, is used to measure the distribution of particle sizes. This method requires the use initially of monodispersed particle reagents in order to detect a change in the anisotropy ratio at low levels of aggregation. The method, however, has a low sensitivity for the detection of aggregates, because only a small change in the anisotropy ratio will be observed. Similarly, the method is primarily useful during the early stages of aggregation where the rate of change of the anisotropy ratio is great.

U.S. Pat. No. 4,521,521, issued to Abbott et al,. discloses a selective particle counting immunoassay that allows the use of nonuniform sized latex particle reagent suspensions. The method combines light-scattering and selective particle counting techniques. Specifically, a stream of an aggregating reaction mixture is passed through an optically defined viewing zone. Focused laser radiation is passed through the viewing zone. A detector, responsive to the intensity of radiation scattered by each particle in the stream, generates an electrical signal related to the size of the particle. A counter network operatively associated with the detector registers a count of the number of particles in the stream that fall within a predetermined size range.

U.S. Pat. Nos. 4,118,192, 4,203,724, and 4,208,185, issued to Sawai et al., describe an agglutination immunoassay that uses antibody- or antigen-sensitized latex particles, with an average diameter not greater than 1.6 microns. The reaction mixture is irradiated with light having a wavelength in the range of 0.6 to 2.4 microns and longer than the average diameter of the carrier particles by a factor of at least 1.5. The absorbance of the reaction mixture is measured as the reaction takes place. When the reaction mixture is irradiated with light that contains rays of polychromatic light having an appropriate wavelength, the absorbance or percent absorption of the reaction mixture increases concomitantly with the progress of the agglutination of the latex.

U.S. Pat. No. 4,766,083, issued to Miyashita et al., discloses a photometric method for measuring a biological agglutination reaction comprising irradiating the reaction mixture with a laser beam and selectively detecting the intensity of scattered light from the reaction system at a scattering angular range (theta) of 30 degrees to 60 degrees. However, the method does not relate particle size distribution to the concentration of an analyte in a sample.

U.S. Pat. No. 5,104,221, issued to Bott et al, discloses a novel method for measuring the size of particles using a Polarization Intensity Differential Scattering (PIDS) technique. The analysis comprises providing an interrogating light beam of selected wavelength, including a first component having a linear polarization plane and a second component having a different linear polarization plane, wherein the linear polarizations of the components are orthogonal. Photodetecting arrays in one or more scattering planes detect light scattered by the particles at least at two scattering angles. The Bott et al. method, however, does not teach using this method for detecting and quantitating analyte concentration in a sample.

There still exists a need for improved methods for quantitating analyte concentration in a sample utilizing particle-based immunoassays that are sensitive, rapid, and can be automated.

### SUMMARY OF THE INVENTION

This invention provides an improved particle-enhanced assay for specifically detecting and quantitatively determining analyte concentration in a sample. The method of this invention provides an improvement over conventional particle-enhanced assays for measuring analyte concentration by improving the particle analysis of the assay reaction mixture. The method involves the determination of the concentration of an analyte in a sample by measuring the relative or absolute distribution of the various particle sizes present in the assay reaction mixture (e.g., individual particle monomers and various clusters of particle monomers) using polarization intensity differential scattering (PIDS) methodology. PIDS is a very sensitive method that effectively allows for the easy discrimination of similar-sized submicron particles. This is achieved by taking advantage of the differences in their scattering patterns when particles of various sizes are irradiated with horizontally and vertically polarized light of different wavelengths. The measurement of the actual distribution of particle sizes can then be correlated with the amount of analyte present in the test sample.

Accordingly, one aspect of this invention provides a direct assay for determining the amount of analyte in a sample, comprising:

a) providing sensitized particles comprising insoluble carrier particles having immobilized thereon a specific binding partner for the analyte;

b) mixing the sample with the sensitized particles in an assay medium to form a reaction mixture;

c) incubating the reaction mixture under conditions that allow the formation of an aggregation reaction mixture comprising said sensitized particles and different sized aggregates thereof,

d) directing a column of light through the reaction mixture, wherein the light comprises a first polarized component and a second polarized component;

e) measuring the distribution of the insoluble sensitized particles and the different sized aggregates in the reaction mixture by polarization intensity differential scattering methodology, wherein the distribution is proportional to the amount of analyte in the sample; and

f) determining the concentration of the analyte in the sample.

The measurement of the particle size distribution is made using polarization intensity differential scattering (PIDS) methodology, which involves sequentially projecting light of various wavelengths and polarization combinations through the reaction mixture, where the light is filtered for polarization in the vertical and horizontal planes, and measuring the average intensity of light scattered by each of the selected configurations of light. The scattered light is detected and converted into data that correlates with the concentration of the analyte of interest in the sample. The combination of multiple wavelengths and two polarizations provides information that differentiates between sub-micron particles of different sizes and increases the resolution and the sensitivity of measuring particle size distributions and hence, analyte concentration measurements.

The process of this invention is suitable for use in any particle-enhanced assay, including direct and competitive assays, for determining the concentration of an analyte in a sample.

### DETAILED DESCRIPTION OF THE INVENTION

The present invention relates to improved particle-enhanced assays for determining the concentration of an analyte in a sample. Briefly, one aspect of this invention provides a direct particle-enhanced assay of an analyte of interest in a sample, wherein the sample is combined with a known amount of sensitized particles comprising insoluble carrier particles having immobilized thereon a plurality of a binding partner for the analyte in an assay medium to form a reaction mixture. The reaction mixture is incubated under conditions that allow the formation of an aggregated reaction mixture comprising said individual sensitized particles and different sized aggregates thereof. The distribution of the individual particles and the various aggregates correlates with the concentration of the analyte originally present in the sample.

A novel feature of this invention comprises an improved method of measuring the distribution of the individual sensitized particles and the different sized aggregates thereof using polarization intensity differential scattering (PIDS) methodology. Data obtained using the PIDS methodology to determine particle size distribution allows for a more accurate determination of analyte concentration in the sample. Thus, the method of this invention provides an improvement over conventional particle-enhanced assays for measuring analyte concentration, primarily by improving particle analysis of the assay reaction mixture. The process of this invention measures the distribution of various particle sizes (i.e., monomer particles as well as dimer, trimer, and N-mer particle aggregates) in a particle-enhanced assay reaction mixture, and is capable of differentiating between particles of similar sizes to provide an extremely sensitive assay.

The term “aggregation” is a process whereby two or more individual sensitized particles are linked together as a result of binding between an analyte and its specific binding partner, one of which is immobilized on the particles, to produce dimer particles, timer particles, and higher order networks of aggregated particles.

For purposes of this invention, the term “particle sizes” includes individual sensitized particle monomers, as well as dimer aggregates, trimer aggregates, and N-mer aggregates thereof, wherein “N” refers to the number of sensitized particles in the aggregate. A “particle monomer” is an individual (i.e., non-aggregated) sensitized particle, i.e., a sensitized particle that is not linked to another sensitized particle.

As used herein, the term “particle size distribution” refers to the distribution of various particle sizes in a particle-enhanced assay reaction mixture. The term “relative distribution” refers to the distribution of the amount of monomers, dimers, trimers, etc. relative to each other.

The term “particle analysis” , as used herein, refers to the measurement of the particle size distribution in a particle-enhanced assay reaction mixture and includes the measurement of the relative percentage or absolute amount of the different particle sizes (e.g., with respect to sample volume or surface area), the change in particle size distribution over time (i.e., rate of aggregation or change in rate of aggregation over time) or the change in other parameters relating to particle size distribution (e.g., peak height, peak width, peak slope or analog or digital responses either before or after mathematical modeling or algorithm manipulations). This measurement correlates with the concentration of analyte in the sample.

As used herein, the term “sensitized particle” refers to an insoluble carrier particle having one or more layers of either an analyte-specific binding partner or the analyte of interest immobilized thereon, where the binding partner is attached to the particle by covalent (chemical) bonds or by non-covalent bonds (e.g., physical adsorption). The carrier particles may be any natural or synthetic material capable of having a binding partner or analyte immobilized thereon. The carrier particles need not be uniform in size. Examples of carrier particles include polymers and copolymers or olefinically unsaturated monomers, such as polystyrene (also referred to as “latex”), acrylonitrile, polybutadiene, and derivatives and copolymers thereof (See, for example, Bangs, L. B., Uniform Latex Particles, (1984) and U.S. Pat. No. 4,305,925), glass, acrylamide, methacrylate, nylon, metals, metal oxides and their derivatives, dextran, cellulose and natural particle material, such as red blood cells, pollens, liposomes, and bacteria.

The term “binding partner” refers to a molecule or substance that specifically recognizes and binds to the analyte of interest, and exhibits negligible cross-reactivity with other substances. Binding between an analyte and its specific binding partner may be via chemical or physical means. Typical binding partners include antigens, antigen fragments, receptors, nucleic acids, and polyclonal and monoclonal antibodies and fragments, derivatives and complexes thereof. Such binding partners specific for a given analyte may be obtained from commercial sources or may be prepared in accordance with standard procedures well known to those skilled in the art.

This invention provides a novel method of measuring particle size distribution or the rate of change of aggregation of a particle-enhanced assay reaction mixture, which in turn allows for improved quantitation of an analyte of interest in a test sample. The method of this invention utilizes the PIDS methodology to perform a particle analysis of a particle-enhanced assay reaction mixture. The scattered light that is detected in the analysis is converted through appropriate modeling and data analysis algorithms into data that correlates with the concentration of the analyte in the sample.

PIDS, or “Polarization Intensity Differential Scattering,” is an enhanced ensemble light-scattering method for the characterization of sub-micron particles down to 0.04 μm. PIDS is described in U.S. Pat. No. 5,104,221, which is specifically incorporated herein by reference. PIDS technology is based on a phenomenon involving the scattering of light of different polarizations and provides a method for measuring small particles at high scattering angles with high resolution. Thus, PIDS effectively allows for the discrimination of different sized submicron particles by taking advantage of the differences in their scattering patterns when the particles are irradiated with horizontally and vertically polarized light of different wavelengths. PIDS analysis can be obtained using appropriate light-scattering instruments, such as the Beckman Coulter LS 230.

Briefly, in a typical PIDS optics system, light from a white light source is filtered through filters of several wavelengths and at two polarizations. The collimated light passing through a sample cell is monitored by a beam monitor. As the sample is introduced to the sample cell, particles in the sample cause the light passing through the cell to scatter from its direct path along the optic axis, and thus the signal detected by the beam monitor decreases. The scattered light is detected by multiple detectors located over a wide angular range, typically from 60 degrees to 145 degrees. The detected signal from each of the detectors is amplified by a dedicated amplifier circuit and is averaged over the duration of the measurement.

When a light beam that is transmitted through an aggregated reaction mixture is oriented in either the vertical (V) direction or the horizontal (H) direction, the detected scattering intensity I_{V} and I_{H} at a given angle will be different. The angular variation of the scattering intensity for the various sized particles is enhanced when the differential intensity between I_{V} and I_{H} (I_{V}-I_{H}) is used instead of I_{V}. The difference between I_{V} and I_{H} (I_{V}-I_{H}) is termed the “PIDS signal.” Since the PIDS signal is dependent on particle size relative to the light wavelength, the measurement of the PIDS signals at several wavelengths advantageously provides additional scattering information that can be used to refine the particle analysis.

In a particle-enhanced assay, the intensity of light scattered by various sized particles is proportional to the number of particles of a given size in the assay reaction mixture at a particular point in time. At the beginning of the assay, the reaction mixture will primarily contain the sensitized particle monomers. As the reaction progresses, however, the monomers come together to form clusters or aggregates of particles, such as dimers, trimers, and N-mers, which are larger in size than the particle monomer. These larger aggregates alter the angular distribution of the scattered intensity, since they act, in effect, like particles of a larger diameter than the individual sensitized particles (monomers). The intensity is proportional to the particle size distribution in the particle-enhanced assay reaction mixture, which is then correlated of the concentration of the analyte of interest in the test sample.

When the measurement is completed, the averaged values obtained at each wavelength and polarization are sequentially written to a data file. The data analysis procedure for the scattering experiments, according to the method of this invention using the PIDS methodology, uses a matrix that includes the PIDS information. The particle size distribution data is representative of analyte concentration in the sample.

As stated above, the PIDS methodology is capable of measuring particle sizes as small as 40 nm. Carrier particles used to prepare the sensitized particles are typically between about 40 nm and 1000 nm. Thus, the method of this invention, which uses the PIDS methodology to measure particle size distribution, offers increased sensitivity over conventional methods. The increased sensitivity is obtained as a result of the PIDS methodology, which measures light-scattering at high angles, and thus offers higher resolution of the particle aggregation size differentiation. Further, the aggregates do not have to be dimers, trimers, etc. formed from particle monomers of the same type or size.

To conduct the assays of the present invention, it is first necessary to provide the sensitized particles. As stated above, the sensitized particles comprise carrier particles having bound thereto either a specific binding partner for the analyte or the analyte of interest. The binding partner or the analyte may be immobilized on the particles in accordance with standard techniques that are well known to those skilled in the art, such as physical (passive) absorption, facilitated (forced) absorption, and covalent coupling. For example, the binding partner or analyte may be covalently attached to the particle surface by modification with chemical functional groups capable of attaching the binding partner to the particle. See, for example, U.S. Pat. Nos. 4,064,080, 4,181,636, 4,210,723, 4,264,766, 4,521,521, and 4,305,925, which are specifically incorporated herein by reference. The techniques disclosed in these patents may be used to attach binding partners or analytes to the particles. See also Seaman G. V. F., ed., Latex Based Technology in Diagnostics, Health & Science Communications, Washington, D.C. 20005 (1990), which is specifically incorporated herein by reference. Sensitized particles having the desired properties and characteristics may also be obtained commercially.

In one embodiment of the method of this invention, a standard curve (i.e., a calibration curve) is first established prior to analyzing a sample for the analyte of interest. That is, a standard quantitative relationship is established between the relative particle size distribution of the particle-enhanced assay reaction mixture as a function of the concentration of the analyte being assayed using known concentration(s) of the sensitized particles. Any number of methods well known to those skilled in the art may be used to prepare the standard curves. (See, Masson et al. and Wilkins et al., supra).

For example, in a direct particle-enhanced assay, several samples containing known amounts of analyte are contacted with its specific binding partner immobilized on insoluble carrier particles. The presence of the analyte in the sample results in crosslinking of the individual particles and thus the formation of particle aggregates of various sizes. As the concentration of the analyte is increased, the extent of aggregation also increases (i.e., an increase in the number of aggregates of a particular size and/or the production of larger sized aggregates). As a way of illustration, four known concentrations of an analyte (0 ng/mL, 0.5 ng/mL, 1 ng/mL, and 10 ng/mL) are added to an optimized assay medium containing known amounts of sensitized particles and the mixtures are incubated to allow formation of aggregated reaction mixtures. The different analyte concentrations correspond to a particle size distribution of 100% (x), [50%(x)−50%(2x)], [100% (2(x)], and [100% (3(x)], respectively, where “x” represents the size (diameter) of the particle monomers in the sample in nanometers (nm). A dose-response curve of analyte concentration versus particle distribution is thus established based on the known concentrations of analyte. An unknown amount of analyte in a sample is then subject to the same reaction conditions. The results obtained for the particle size distribution are then compared to the standard curve to determine the concentration of analyte in the sample.

For the purpose of generating the standard curve, the relative particle size distribution can be characterized in several ways, such as by measuring the ratio of particle monomers, dimers, trimers, etc., per unit volume of sample. The standard curve can be employed as a standard to be applied when performing particle-enhanced assays on samples containing unknown amounts of the analyte of interest.

To perform a direct particle-enhanced assay of this invention, sensitized particles comprising an immobilized analyte-specific binding partner are provided. A sample suspected of containing an analyte of interest is combined with a known amount of the sensitized particles in an assay medium to form a reaction mixture. The reaction mixture is incubated under conditions that allow binding to occur between the analyte present in the sample and the immobilized binding partner, thus forming an aggregated reaction mixture. Methods of preparing and incubating such reaction mixtures are well known to those skilled in the art and need not be described in further detail. Once a measurable reaction has proceeded, the particle size distribution in the reaction mixture is measured by the PIDS methodology. In one embodiment, the reaction mixture is flowed through a reaction flow cell that flows the reaction mixture past a beam of light that is polarized in the horizontal and vertical planes relative to the scattering plane. In this embodiment, a PIDS measurement is generated by continuously pumping the reaction mixture through the reaction flow cell at a rate such that all, or a substantial portion, of the particle-containing sample passes through the reaction cell where the scattered light measurement is made. Alternatively, the sample, the sensitized particles, and the assay medium are combined and incubated in the reaction cell and the PIDS measurement is taken on the reaction mixture contained in the reaction cell.

The PIDS measurement is made according to the method disclosed in U.S. Pat. No. 5,104,221, which is specifically incorporated herein by reference. In general, the PIDS measurement is made by sequentially projecting a light beam of several wavelengths and polarization combinations through the reaction cell, and measuring the average intensity of light scattered at various angles to the axis of the beam. The amplitude and angle of the scattered light is, in part, a function of particle size. Portions of light scattered by the particles in the reaction mixture pass through collection lenses to the focal surfaces of the various detectors. As a result, each location on focal surfaces of the detectors receives light scattered at a single, unique angle to the input axis of the beam in the reaction cell. By measuring light intensity at small discrete locations at the focal surfaces, the intensity/angle profile of light scattered by particles in the reaction cell can be determined. In one embodiment, the light comprises three different wavelengths, each comprising two polarization combinations, and the scattered light is measured for a time period of between 10 and 30 seconds. Thus, most of the particles in the sample system are sensed with each variety of projected light.

When the measurement is completed, the averaged values obtained at each wavelength and polarization are sequentially written to a data file. The averaged values are then converted into data representative of the particle size distribution through appropriate modeling and data analysis algorithms. The particle size distribution data is compared with a standard curve to determine the original concentration of the analyte in the test sample.

By using the PIDS methodology to measure particle size distribution, the method of this invention provides an improved particle-enhanced assay which is highly sensitive and effective in accurately measuring extremely low concentrations, such as 10⁻¹¹ to 10⁻⁸ grams per milliliter of analyte in a sample.

In general, one aspect of this invention provides a direct assay which comprises contacting a sample which may contain the analyte of interest with particles sensitized with analyte-specific binding partners to form a reaction mixture, incubating the reaction mixture under conditions that allow binding between the analyte and the particle-bound binding partners, wherein the binding causes aggregation of the particles, and measuring the particle size distribution using the PIDS methodology. Any conditions that will allow the formation of aggregated particles may be used in the method of this invention and are well known to those skilled in the art. The extent of particle aggregation is directly proportional to the concentration of analyte in the sample.

Another aspect of this invention provides a competitive assay for the determination of an analyte in a sample. In this assay, a known quantity of binding partner is added to the test sample containing the analyte of interest. This mixture then is combined with sensitized particles comprising carrier particles having analyte bound thereto. The analyte present in the test sample and the immobilized analyte react competitively with the binding partner. The greater the amount of analyte present in the test sample, the less binding partner available for linking sensitized particles to induce aggregation, and the greater the inhibition of particle aggregation. The degree to which the binding partner inhibits the aggregation of the sensitized particles indicates the concentration of analyte present in the sample. In this assay, the extent of particle aggregation is inversely proportional to the concentration of analyte in the sample.

This invention can be applied to the detection and measurement of any analyte of interest for which a specific binding partner exists or for which a specific binding partner may be prepared. As used herein, an “analyte” refers to the substance whose presence and/or concentration in a sample is to be determined. The term “analyte” refers to any substance for which there exists a specific binding partner, or for which a specific binding member can be prepared, and which will bind the specific binding member in an assay. Representative analytes include, but are not limited to, antigens, haptens, antibodies, proteins, peptides, amino acids, hormones, steroids, cancer cell markers, tissue cells, viruses, vitamins, drugs, metabolites, nucleic acids, and pesticides. Analytes of interest that may be measured in accordance with the present invention include any analyte which is capable of promoting or inhibiting a particle-enhanced reaction upon contact or reaction with a binding partner that is immobilized on an insoluble particle.

The term “antibody”, as used herein, refers to immunoglobulins that are produced in response to the detection of a foreign substance, and includes intact molecules as well as functional fragments thereof, such as Fab, F(ab′)₂, and Fv. These antibody fragments retain some ability to selectively bind with their antigen or receptor.

As used herein, the term “sample” includes, but is not limited to, biological fluids, such as blood, plasma, serum, saliva, cerebral spinal fluid, urine, and any other type of fluid, cells, tissues or material which is suspected of containing an analyte of interest. By “cells,” it is meant any sample obtained from a bodily organ or other tissue, e.g., a tumor, from which a cellular suspension can be prepared. The sample may be diluted or undiluted, and may be untreated or treated, according to methods well known to those skilled in the art. Such treatments include pH adjustment, separation (filtration, centrifugation, etc.), chemical treatment with enzymes and/or other chemicals, physical treatment (homogenization, sonication, etc).

In one embodiment, the apparatus useful for the invention includes:

(a) a sample cell for holding a reaction mixture obtained by reacting the sensitized particles and a sample containing the analyte of interest;

(b) means for illuminating the sample cell along a first axis with one or more light beams, each light beam characterized by a selected wavelength and including

(i) at least a first component having a linear polarization at a first angle measured with respect to the axis and

(ii) a second interrogating component having a linear polarization at a second angle measured with respect to the axis, where the first and second angles are other than symmetrically disposed about a plane perpendicular or parallel to the scattering plane and intersecting the axis;

(c) photodetector means for detecting light scattered by the various sized particles in the reaction mixture, the photodetector means including means for generating a first intensity signal representative of the intensity of scattered light from the first component, the photodetector means including means for generating a second intensity signal representative of the intensity of scattered light from the second component; and

(d) intensity differential processing means, coupled to the photodetector means, for generating a signal representative of the particle size distribution in the sample cell for at least one selected wavelength, the intensity differential processing means including means for generating a difference signal representative of the difference of the first and second intensity signals.

The method of this invention has important advantages over immunoassays that utilize conventional light-scattering methods. For example, during the progress of a particle-enhanced assay reaction, aggregates of sensitized particles grow, forming dimers, trimers, and N-mers, which are larger in size than the particle monomer. These larger complexes alter the angular distribution of the scattered light, since they act, in effect, like particles of a larger diameter than the original sensitized particles (monomers). However, large particles scatter light at small angles, and relatively large changes in their size produce only small changes in the scattering pattern. The method of this invention overcomes this drawback of prior art methods, since the PIDS methodology, which measures light scattered at high angles with high resolution, provides a more sensitive means in detecting small changes in the scattering pattern when compared to conventional techniques. Thus, employing the PIDS methodology to measure particle size distribution provides a more sensitive means of measuring the amount of analyte of interest in a sample.

The invention may be embodied in other specific forms without departing from its essential characteristics. The described embodiments are to be considered in all respects only as illustrative and not as restrictive. Indeed, those skilled in the art can readily envision and produce further embodiments, based on the teachings herein, without undue experimentation. The scope of the invention is, therefore, indicated by the appended claims rather than by the foregoing description. All changes that come within the meaning and range of the equivalence of the claims are to be embraced within their scope.

We claim:

1. A method for determining the concentration of an analyte in a sample, comprising:
a) mixing a sample comprising an unknown concentration of said analyte with a known amount of sensitized particles in an assay medium to form a reaction mixture, wherein said sensitized particles comprise carrier particles having immobilized thereon either said analyte of interest or a binding partner for said analyte;
b) incubating said reaction mixture under conditions that allow the formation of an aggregation reaction mixture comprising said individual sensitized particles and different sized aggregates thereof,
c) determining the distribution of said individual sensitized particles and said different sized aggregates thereof in said reaction mixture by polarization intensity differential scattering methodology; and
d) determining the concentration of said analyte in said sample.

2. The method of claim 1, wherein said determining includes comparing said particle size distribution obtained in step (c) with a calibration curve.

3. The method of claim 1, wherein said determining includes measuring said particle size distribution as a function of time, wherein the change in the distribution of aggregate size as a function of time is utilized to determine the amount of said analyte in said sample.

4. The method of claim 1, wherein said sensitized particles are carrier particles having immobilized thereon a binding partner that specifically binds said analyte.

5. The method of claim 4, wherein the presence of said analyte in said sample promotes particle aggregation.

6. The method of claim 1, wherein said sensitized particles are carrier particles having immobilized thereon said analyte, wherein said method further comprises adding a known amount of an analyte-specific binding partner to said reaction mixture.

7. The method of claim 6, wherein the presence of said analyte in said sample inhibits particle aggregation.

8. The method of claim 1, wherein said insoluble particles are selected from the group consisting of polystyrene, acrylonitrile, polybutadiene, acrylamide, methacrylate, nylon, metals, metal oxides and their derivatives, glass, dextran, cellulose, red blood cells, pollens, liposomes, and bacteria.

9. The method of claim 8, wherein said insoluble particles are polystyrene.

10. The method of claim 1, wherein said binding partner or said analyte is supported on said insoluble sensitized particles by covalent or non-covalent bonds.

11. The method of claim 1, wherein said analyte is selected from the group consisting of antigens, haptens, antibodies, proteins, peptides, amino acids, hormones, steroids, cancer cell markers, tissue cells, viruses, vitamins, drugs, metabolites, nucleic acids, and pesticides.

12. The method of claim 1, wherein said binding partner is selected from the group consisting of antigens, antigen fragments, receptors, nucleic acids, monoclonal antibodies, polyclonal antibodies, and antibody fragments.

13. The method of claim 1, wherein said sample is selected from the group consisting of blood, plasma, serum, saliva, cerebral spinal fluid, urine, cells, and tissues.

14. The method of claim 1, wherein said polarization intensity differential scattering methodology comprises:
a) illuminating the reaction mixture along an axis with one or more light beams, each light beam characterized by a selected wavelength and including
i) at least a first component having a linear polarization at a first angle measured with respect to the axis, and
ii) a second component having a linear polarization at a second angle measured with respect to the axis, wherein the first and second angles are other than symmetrically disposed about a plane perpendicular or parallel to the scattering plane and intersecting the axis;
b) providing photodetector means for detecting light scattered by said reaction mixture, the photodetector means including
i) means for generating a first intensity signal representative of the intensity of scattered light from the first component, and
ii) means for generating a second intensity signal representative of the intensity of scattered light from the second component; and
c) providing intensity differential processing means, coupled to said photodetector means, for generating a signal representative of the particle size distribution in said reaction cell for at least one selected wavelength, said intensity differential processing means including means for generating a difference signal representative of the difference of said first and second intensity signals.

15. A method for determining the concentration of an analyte of interest in a sample, comprising:
a) providing a select amount of sensitized particles comprising insoluble carrier particles having immobilized thereon either said analyte of interest or a binding partner that specifically binds said analyte;
b) mixing said sample with said sensitized particles in an assay medium to form a reaction mixture;
c) incubating said reaction mixture under conditions that allow the formation of an aggregation reaction mixture comprising said individual sensitized particles and different sized aggregates thereof;
d) directing a column of light through said reaction mixture, wherein the light comprises a first polarized component and a second polarized component;
e) measuring the size distribution of said individual sensitized particles and said different sized aggregates thereof in said reaction mixture by polarization intensity differential scattering methodology, wherein the distribution is related to the amount of analyte in the sample; and
f) determining the concentration of said analyte in said sample.

16. The method of claim 15, wherein said determining comprises comparing the size distribution with standard curve data representing the concentration of said analyte of interest as a function of size distribution, wherein said comparing provides a quantitative measure of the concentration of said analyte of interest in said sample.

17. The method of claim 15, wherein said sensitized particles comprise insoluble particles having immobilized thereon an analyte-specific binding partner.

18. The method of claim 15, wherein said sensitized particles comprise insoluble particles having immobilized thereon said analyte, wherein said method further comprises adding a known amount of analyte-specific binding partner to said reaction mixture.

19. The method of claim 15, wherein said insoluble particles are selected from the group consisting of polystyrene, acrylonitrile, polybutadiene, acrylamide, methacrylate, nylon, metals, metal oxides and their derivatives, glass, dextran, cellulose, red blood cells, pollens, liposomes, and bacteria.

20. The method of claim 19, wherein said insoluble particles are polystyrene.

21. The method of claim 15, wherein said binding partner is selected from the group consisting of antigens, antigen fragments, receptors, nucleic acids, monoclonal antibodies, polyclonal antibodies, and antibody fragments.

22. The method of claim 15, wherein said binding partners are immobilized on said insoluble particles by covalent or non-covalent bonds.

23. The method of claim 15, wherein said analyte is selected from the group consisting of antigens, haptens, antibodies, proteins, peptides, amino acids, hormones, steroids, cancer cell markers, tissue cells, viruses, vitamins, drugs, metabolites, nucleic acids, and pesticides.

24. The method of claim 15, wherein said sample is selected from the group consisting of blood, plasma, serum, saliva, cerebral spinal fluid, urine, cells, and tissues.

25. A method of measuring the concentration of an analyte in a sample, comprising:
a) providing a select amount of sensitized particles comprising insoluble particles having immobilized thereon either said analyte of interest or a binding partner that specifically binds said analyte;
b) mixing said sample with said sensitized particles in an assay medium to form a reaction mixture;
c) incubating said reaction mixture under conditions that allow the formation of an aggregation reaction mixture comprising said individual sensitized particles and different sized aggregates thereof;
d) sequentially projecting the light of multiple wavelengths along an axis through said reaction mixture, wherein the light is filtered for polarization in the vertical and horizontal planes relative to said axis;
e) measuring the average intensity of light scattered by the vertically polarized light to generate a first intensity signal;
f) measuring the average intensity of light scattered by the horizontally polarized light to generate a second intensity signal;
g) generating a difference signal representative of the difference of said first and second intensity signals; and
h) processing the difference signal to generate a resultant signal representative of the distribution of said individual sensitized particles and said different sized aggregates thereof in said reaction mixture; and
i) comparing the resultant signal to a standard curve, whereby the concentration of said analyte in said sample is measured.

26. The method of claim 25, wherein said determining comprises comparing the size distribution with standard curve data representing the concentration of said analyte of interest as a function of size distribution, wherein said comparing provides a quantitative measure of the concentration of said analyte of interest in said sample.

27. The method of claim 25, wherein said sensitized particles comprise insoluble particles having immobilized thereon an analyte-specific binding partner.

28. The method of claim 25, wherein said sensitized particles comprise insoluble particles having immobilized thereon said analyte, wherein said method further comprises adding a known amount of analyte-specific binding partner to said reaction mixture.

29. The method of claim 25, wherein said binding partner or said analyte is immobilized on said insoluble sensitized particles by covalent or non-covalent bonds.

30. The method of claim 25, wherein said sample is selected from the group consisting of blood, plasma, serum, saliva, cerebral spinal fluid, urine, cells, and tissues.

31. The method of claim 25, wherein said analyte is selected from the group consisting of antigens, haptens, antibodies, proteins, peptides, amino acids, hormones, steroids, cancer cell markers, tissue cells, viruses, vitamins, drugs, metabolites, nucleic acids, and pesticides.

32. The method of claim 25, wherein said binding partner is selected from the group consisting of antigens, antigen fragments, receptors, nucleic acids, monoclonal antibodies, polyclonal antibodies, and antibody fragments.

33. The method of claim 25, wherein the insoluble particles are selected from the group consisting of polystyrene, acrylonitrile, polybutadiene, acrylamide, methacrylate, nylon, metals, metal oxides and their derivatives, glass, dextran, cellulose, red blood cells, pollens, liposomes, and bacteria.

34. The method of claim 33, wherein said particles are polystyrene.

35. The method of claim 25, wherein said scattered light is measured by multiple detectors positioned at about 90 degrees to the direction of the light path, wherein said detectors measure the differential intensity between scattered light of vertical and horizontal polarizations.
